(19) United States
(12) Patent Application Publication
(10) Pub. No.: US 2023/0181919 A1
(43) Pub. Date: Jun. 15, 2023

(54) TRANSDUCER ARRAY WITH SHAPE THAT CONTOURS TO A SUBJECT'S BODY AND METHOD OF DETERMINING SHAPE AND PLACEMENT OF TRANSDUCER ARRAYS

(71) Applicant: Novocure GmbH, Root D4 (CH)

(72) Inventors: Darja WENDEL, Lisbon (PT); Woonghee HAN, San Francisco, CA (US)

(73) Assignee: Novocure GmbH, Root D4 (CH)

(21) Appl. No.: 18/080,306

(22) Filed: Dec. 13, 2022

Related U.S. Application Data
(60) Provisional application No. 63/289,523, filed on Dec. 14, 2021.

(51) Int. Cl. A61N 1/40

(52) U.S. Cl. CPC A61N 1/40

(57) ABSTRACT

A transducer apparatus for delivering tumor treating fields to a subject's body, the transducer apparatus including: a substrate and an array of electrodes disposed on the substrate, the array configured to be positioned over the subject's body with a face of the array facing the subject's body; wherein, when viewed from a direction perpendicular to the face of the array, the substrate has at least one concave edge forming at least a portion of a boundary of the array, the at least one concave edge defines an opening between two opposing sides of the substrate, no electrodes are present in the opening, and the substrate has a substantially C-shaped, U-shaped, rounded V-shaped, or annular shaped surface.

### CROSS REFERENCE TO RELATED APPLICATIONS

This application claims priority to U.S. Provisional Patent Application No. 63/289,523, filed Dec. 14, 2021, which is hereby incorporated by reference in its entirety.

### BACKGROUND

Tumor treating fields (TTFields) are low intensity alternating electric fields within the intermediate frequency range, which may be used to treat tumors as described in U.S. Pat. No. 7,565,205. TTFields are induced non-invasively into the region of interest by transducers placed on the patient's body and applying AC voltages between the transducers. Conventionally, transducers used to generate TTFields include a plurality of ceramic disks. One side of each ceramic disk is positioned against the patient's skin, and the other side of each disc has a conductive backing. Electrical signals are applied to this conductive backing, and these signals are capacitively coupled into the patient's body through the ceramic discs. Conventional transducer designs include rectangular arrays of ceramic disks aligned with each other in straight rows and columns (e.g., in a three-by-three arrangement) and attached to the subject's body via adhesive.

### SUMMARY OF THE INVENTION

One aspect of the invention is directed to a transducer apparatus for delivering tumor treating fields to a subject's body. The transducer apparatus includes a substrate and an array of electrodes disposed on the substrate. The array is configured to be positioned over the subject's body with a face of the array facing the subject's body. When viewed from a direction perpendicular to the face of the array, the substrate has at least one concave edge forming at least a portion of a boundary of the array, the at least one concave edge defines an opening between two opposing sides of the substrate, no electrodes are present in the opening, and the substrate has a substantially C-shaped, U-shaped, rounded V-shaped, or annular shaped surface.

The above aspect of the invention is exemplary, and other aspects and variations of the invention will be apparent from the following detailed description of embodiments.

### BRIEF DESCRIPTION OF THE DRAWINGS

FIGS. 1A and 1B depict an example of transducers located on a subject's torso.

FIGS. 2A and 2B depict another example of transducers located on a subject's torso.

FIG. 3 depicts an example of transducers located on a subject's head.

FIGS. 4A-4G depict example layouts of an array of electrode elements on a transducer apparatus.

FIGS. 5A and 5B are cross-sectional views of examples of the structure of various transducers.

FIG. 6 depicts an example of a configuration of one pair of transducers.

FIG. 7 depicts an example of an apparatus to determine locations and shapes of transducers on a subject's body for applying TTFields.

FIG. 8 is a flowchart depicting an example of determining locations and shapes of transducers on a subject's body for applying TTFields.

### DESCRIPTION OF EMBODIMENTS

As used herein, a substrate having “a substantially C-shaped, U-shaped, rounded V-shaped, or annular shaped surface” means a substrate having “a substantially C-shaped, a substantially U-shaped, a substantially rounded V-shaped, or a substantially annular shaped surface”. In each case, the term “substantially” includes shapes generally recognized as and functionally equivalent to the described shape. For example, the term “substantially U-shaped” includes a rounded flat-bottomed U-shape, and the term “substantially C-shaped” includes a rounded straight-edged C-shape. The two opposing sides, or arms, of the U-shape need not be perfectly parallel and need not be straight. Further, as described herein, the annular shapes of a substrate, such as circular, oval, ovaloid, ovoid, elliptical, etc., (for which at least one concave edge defines an interior opening bounded on all sides by the substrate) may include a slit (from an exterior boundary of the substrate to the interior opening) which may allow at least temporary access to the interior opening.

This application describes exemplary transducer apparatuses used to apply TTFields to a subject's body for treating one or more cancers. This application also describes exemplary methods to determine the shape and placement of transducer arrays on a subject's body for applying TTFields to the subject's body.

Transducers used to apply TTFields to a subject's body often include multiple electrode elements coupled together on a substrate and attached to the subject's body at a desired location, for example, via an adhesive backing of the substrate or a separately applied adhesive. Conventional transducers have relatively large, rectangular, planar surfaces so as to maximize a number of ceramic electrode elements that are located on the transducer and used to apply TTFields to the subject's body. However, the flat rectangular design limits the flexibility of the transducer for contouring to many parts of the subject's body. In addition, if areas on the subject's body are to be avoided by the electrode elements, practitioners have had to place the transducer at a suboptimal location of the subject's body for applying the desired TTFields.

The inventors have now recognized that a need exists for transducers with greater flexibility for comfortably contouring to a subject's body. In addition, the inventors have now recognized that a need exists for transducers that can easily fit around certain anatomical features, chemotherapy ports, and/or other areas to be avoided on the subject's body. Transducers that are able to effectively contour to a subject's body while avoiding areas that need to remain uncovered and/or would otherwise cause discomfort can be placed at an optimal location on the subject's body. As a result, the transducers can induce TTFields through the subject's body at an ideal location and power level for targeting a region of interest (e.g., tumor) in the subject's body, thereby improving patient outcomes.

The disclosed transducer apparatuses have a substantially C-shaped, U-shaped, rounded V-shaped, or annular shaped surface. The disclosed transducer apparatuses may be readily contoured to the anatomical shape of the subject's body without needing to be reconfigured or placed in a sub-optimal location. The shapes of the disclosed transducer apparatuses provide greater flexibility and continuity for transducer placement around areas such as a breast, ear, chemotherapy port, surgical scar, skin lesion, or any other shaped area on the subject's body that is difficult for the placement of transducers without causing discomfort. In addition, the uniquely shaped transducer apparatuses may be pre-selected by transducer placement planning software in accordance with the disclosed methods. For example, the particular location and shape of the transducers may be chosen together, thereby providing an improved transducer layout to apply a maximum amount of TTFields to the region of interest in the subject's body.

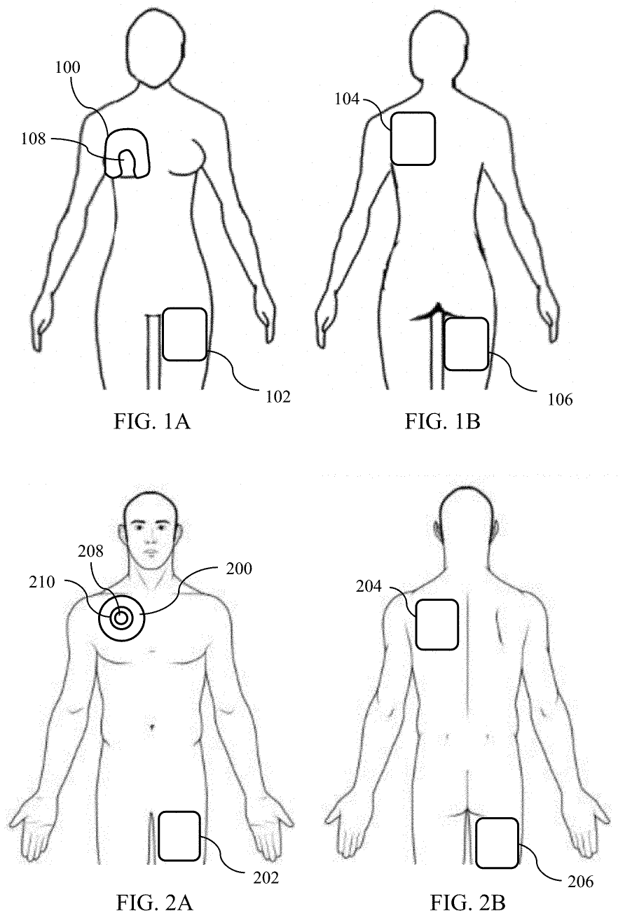

FIGS. 1A and 1B depict an example of transducers positioned at locations on a subject's torso. FIG. 1A depicts a first transducer 100 located on the front of the subject's right breast and a second transducer 102 located on the front of the subject's left thigh. FIG. 1B depicts a third transducer 104 located on the left side of the subject's upper back and a fourth transducer 106 located on the back of the subject's right thigh. The transducers 100, 102, 104, and 106 are transducer arrays each including a plurality of electrodes located on a surface that is flexible for contouring to the subject's body.

Similarly, FIGS. 2A and 2B depict another example of transducers positioned at locations on a subject's torso. FIG. 2A depicts a first transducer 200 located on the front of the subject's right thorax and a second transducer 202 located on the front of the subject's left thigh. FIG. 2B depicts a third transducer 204 located on the left side of the subject's upper back and a fourth transducer 206 located on the back of the subject's right thigh. The transducers 200, 202, 204, and 206 are transducer arrays each including a plurality of electrodes located on a surface that is flexible for contouring to the subject's body.

Transducers arranged on a subject's torso (as shown in FIGS. 1A-2B) are capable of applying TTFields to a tumor in the subject's thorax or abdomen. The transducers may be located at various other combinations of locations on the subject's torso than those of FIGS. 1A-2B.

Both FIGS. 1A/1B and FIGS. 2A/2B illustrate an assembly for applying TTFields to a subject's body while avoiding at least one area with an anatomic feature or device. For example, in FIG. 1A, the surface of the transducer 100 is substantially U-shaped for contouring over a breast of the subject's body while avoiding a nipple 108 of the subject's body. In another example, the surface of the transducer 100 may be substantially C-shaped, rounded V-shaped, or annular shaped for contouring over the breast while avoiding the nipple 108 of the subject's body.

As another example, in FIG. 2A, the surface of the transducer 200 is shaped to avoid a chemotherapy port 208 on the subject's body. In particular, the surface of the transducer 200 is substantially annular shaped and is adapted to be positioned on the subject's body such that an interior opening 210 of the transducer 200 coincides with a location on the subject's body having the chemotherapy port 208. In another example, the surface of the transducer 200 may be substantially C-shaped, U-shaped, or rounded V-shaped and adapted to be positioned on the subject's body with two opposing portions of the transducer surface spaced apart to straddle a location on the subject's body having the chemotherapy port 208. In both examples, no electrodes of the transducer 200 are located over the chemotherapy port 208. Chemotherapy ports 208 are often inserted into a subject's body prior to the subject receiving TTFields treatment. The transducers disclosed herein may enable the application of TTFields to a region of interest in the subject's thorax or abdomen without interfering with or being affected by the subject's chemotherapy port 208.

Turning back to FIGS. 1A and 1B, one or more other transducers 102, 104, and 106 may have a different shape than the transducer 100. As illustrated, for example, each of the second, third, and fourth transducers 102, 104, and 106 of the assembly has a different shape than the first transducer 100. In some embodiments, each of the second, third, and fourth transducers 102, 104, and 106 may have the same or a substantially similar shape to each other. As illustrated, the surface of at least one of the transducers 102, 104, and 106 may have a substantially convex shape. More particularly, the surface of at least one of the transducers 102, 104, and 106 may have a rectangular, substantially rectangular with rounded corners (as illustrated), circular, oval, ovaloid, ovoid, or elliptical shape. The transducers 202, 204, and 206 illustrated in FIGS. 2A and 2B have similar shapes as the transducers 102, 104, and 106 shown in FIGS. 1A and 1B.

In other embodiments, one or more of the other transducers 102, 104, and 106 may have a surface having the same shape or a mirror image shape of the transducer 100 of FIGS. 1A and 1B. For example, two transducers may have the same substantially U-shape for contouring over both breasts of the subject's body while avoiding both nipples. Similarly, one or more of the other transducers 202, 204, and 206 may have a surface having the same shape or a mirror image shape compared to the transducer 200 of FIGS. 2A and 2B.

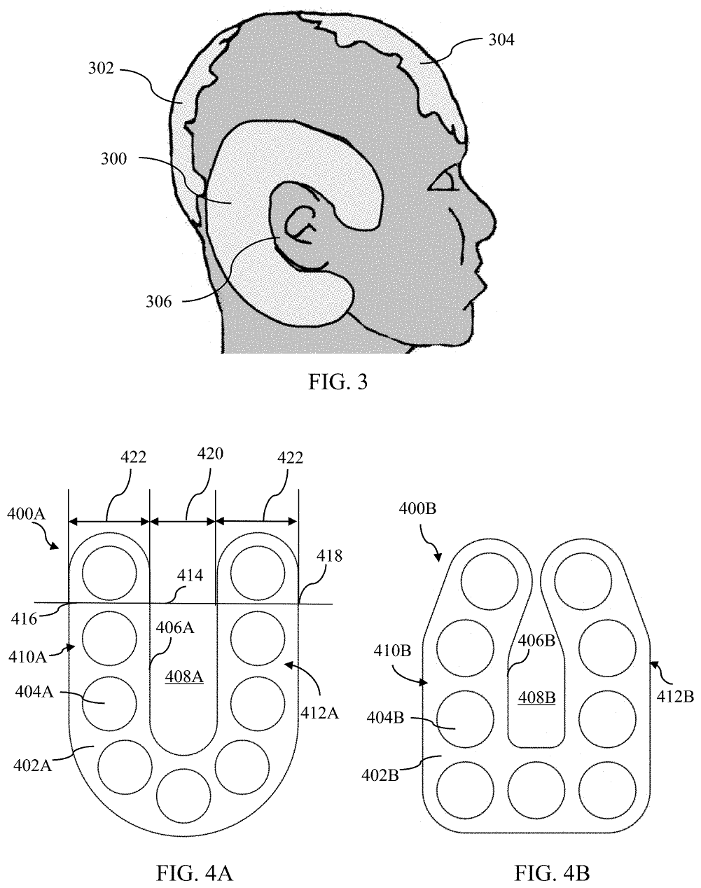

FIG. 3 depicts an example of transducers positioned on the head of a subject's body. FIG. 3 depicts one example of a subject's head on which transducers 300, 302, and 304 are placed in various positions and/or orientations. The transducers 300, 302, and 304 are transducer arrays each including a plurality of electrodes located on a surface that is flexible for contouring to the subject's body. Although not shown in the present view of the subject's head, a fourth transducer may be located on an opposite side of the subject's head from the first transducer 300. Transducers arranged on a subject's head (as shown in FIG. 3) are capable of applying TTFields to a tumor in a region of the subject's brain. The transducers may be located at various other combinations of locations on the subject's head than those of FIG. 3.

FIG. 3 illustrates an assembly for applying TTFields to a subject's body while avoiding at least one area with an anatomic feature or device. For example, in FIG. 3, the surface of the transducer 300 is substantially C-shaped for contouring over the subject's head while avoiding an ear 306 of the subject's body. In another example, the surface of the transducer 300 may be substantially U-shaped, rounded V-shaped, or annular shaped for contouring over the subject's head while avoiding the ear 306 of the subject's body.

One or more other transducers 302 and 304 may have a different shape than the transducer 300. As illustrated, for example, each of the second and third transducers 302 and 304 of the assembly has a different shape than the first transducer 300. In some embodiments, each of the second (302), third (304), and fourth (not shown) transducers may have the same or a substantially similar shape to each other. The surface of at least one of the transducers 302 and 304 may have a substantially convex shape. More particularly, the surface of at least one of the transducers 302 and 304 may have a rectangular, substantially rectangular with rounded corners, circular, oval, ovaloid, ovoid, or elliptical shape. As illustrated, the surface of the at least one of the transducers 302 and 304 may have a scalloped edge.

In other embodiments, one or more of the transducers 302, 304, or a fourth transducer (not shown) may have a surface having the same shape or a mirror image shape of the transducer 300 of FIG. 3. For example, a fourth transducer located opposite the first transducer 300 may have a mirror image of the C-shape of the transducer 300 for contouring over the subject's head while avoiding the other ear of the subject's body.

As disclosed herein, various shapes of transducers may be used to avoid at least one area of the subject's body. The transducers may include, for example, a substantially C-shaped, a substantially U-shaped, a substantially rounded V-shaped, or a substantially annular shaped transducer. In addition, the transducers may be located on the subject's body relative to one or more areas to avoid on the subject's body including, for example, a breast of the subject's body, a nipple of the subject's body, an ear of the subject's body, an eye of the subject's body, a surgical scar or skin lesion of the subject's body, an armpit of the subject's body, or a chemotherapy port disposed in the subject's body.

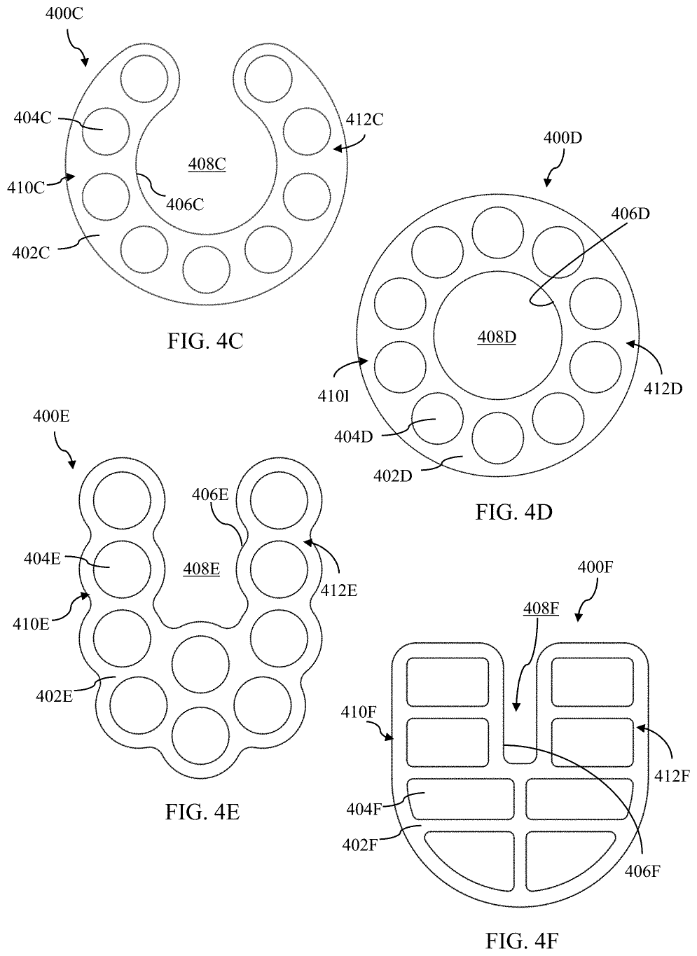

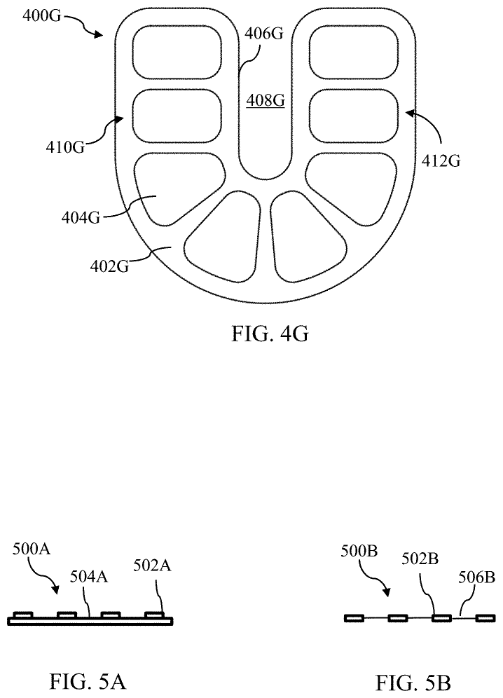

FIGS. 4A-4G depict example transducer apparatuses in accordance with the present disclosure. In each of FIGS. 4A-4G, the transducer apparatus (e.g., 400A, 400B, 400C, 400D, 400E, 400F, and 400G) includes a substrate (e.g., 402A, 402B, 402C, 402D, 402E, 402F, 402G) and an array of electrodes (e.g., 404A, 404B, 404C, 404D, 404E, 404F, 404G) on the substrate (402A-G). The array of electrodes (404A-G) is configured to be positioned over the subject's body with a face of the array facing the subject's body. FIGS. 4A-4G each illustrate a transducer apparatus (400A-G) as viewed from a direction perpendicular to this face of the array of electrodes (404A-G). For all embodiments, the electrodes may be of any shape; and for any given embodiment, the electrodes may all be of the same shape, or one or more electrodes may differ in size and/or shape from other electrodes. In each of FIGS. 4A-4G, the substrate (402A-G) has at least one concave edge (e.g., 406A, 406B, 406C, 406D, 406E, 406F, 406G) forming at least a portion of a boundary of the array. The at least one concave edge (406A-G) may define an opening (e.g., 408A, 408B, 408C, 408D, 408E, 408F, 408G) between two opposing sides (e.g., 410A/412A, 410B/412B, 410C/412C, 410D/412D, 410E/412E, 410F/412F, 410G/412G) of the substrate (402A-G). No electrodes (404A-G) are present in the opening (408A-G) in FIGS. 4A-4G. In addition, there may be no hydrogel or adhesive present in the opening (408A-G). The transducers 400A-G may be placed on a subject's body such that the opening (408A-G) is positioned over an area to avoid on the subject's body.

Although the substrate shapes are illustrated in the Figures herein using perfect curves and straight lines, it is to be understood that the terms substantially C-shaped, substantially U-shaped, substantially rounded V-shaped, and substantially annular shaped include all such general shapes; and the substrate shapes may be formed using irregular curves, curves formed from a series of straight lines or arcs of differing radius of curvature, scalloped edges (for example, tracing the outline of one or more individual electrode shapes), or non-parallel edges. Furthermore, the ends of the two arms of a substrate outline (for example, the arms of a U-shape, rounded V-shape, or C-shape, etc.) that define an opening may be rounded (curved, as shown, for example, in FIG. 4A, FIG. 4B, FIG. 4C and FIG. 4E) or may be straight (flattened curves, as shown, for example, in FIG. 4F and FIG. 4G).

FIG. 4A illustrates a substrate 402A having a substantially U-shaped surface. For a perfect U-shape, the inner concave boundary has a perfectly curved arc and represents exactly one half of a circle before each inner edge boundary of the two arms straightens to form the U-shape (each arm deviating from the parallel position by 0 degrees). The U-shape may incorporate any radius of curvature and therefore the U-shape may be of any size. In some embodiments, the two opposing sides 410A and 412A of the substrate 402A may be substantially straight and extend in substantially parallel directions (e.g., each arm deviating from the parallel position by 2 degrees or less). In some embodiments, the two opposing sides 410A and 412A of the substrate 402A may be substantially straight and extend in substantially parallel directions (e.g., each arm deviating from the parallel position by 20 degrees or less). In such an embodiment, the two arms may align slightly toward each other or align slightly away from each other. In embodiments where the arms are aligned toward each other (not shown), they may not overlap, although they may almost come together, leaving a slight opening. Particularly when the substrate is formed from a flexible material, the latter configuration may allow affixing the transducer apparatus to the body while maneuvering and positioning around a chemotherapy port which is already in place on the body. In embodiments where the two arms are aligned away from each other (not shown), this shape may also be viewed as a rounded V-shape. In some embodiments, the two opposing sides 410A and 412A of the rounded V-shape substrate 402A may be substantially straight and extend away from each other (e.g., each arm deviating from the parallel position by less than 45 degrees, but also by more than or equal to 20 degrees). FIG. 4A also illustrates a spatial arrangement between the opening 408A and opposing sides 410A/412A. Specifically, a straight line 414 is drawn through the two opposing sides 410A and 412A of the substrate 402A, from an outer substrate boundary 416 to an opposing outer substrate boundary 418, and across the opening 408A. A distance 420 along the line 414 across the opening 408A may be at least 25% of a distance 422 along the line 414 across one of the opposing sides 410A and 412A of the substrate 402A. In some embodiments, the distance 420 across the opening 408A may be at least 50% of a distance 422 across one of the opposing sides 410A and 412A. In some embodiments, the distance 420 across the opening 408A may be 100% or more of a distance 422 across one of the opposing sides 410A and 412A. This spatial arrangement between the opening (408) and the opposing sides (410/412) may be similar among the transducers apparatuses of FIGS. 4B-4G as well. In each case, the distance 420 along the line 414 is taken at the widest part across the opening 408.

FIG. 4B illustrates a substantially U-shaped substrate 402B having a flat-bottomed U-shaped surface, wherein the two opposing sides (arms) 410B and 412B of the substrate 402B are aligned slightly toward each other. In another embodiment, the substantially U-shaped substrate 402B has a flat-bottomed U-shaped surface, wherein the two opposing sides (arms) 410B and 412B of the substrate 402B are aligned parallel to each other. In another embodiment, the substantially U-shaped substrate 402B has a flat-bottomed U-shaped surface, wherein the two opposing sides (arms) 410B and 412B of the substrate 402B are aligned slightly away from each other as discussed above for the rounded U-shaped embodiments.

FIG. 4C illustrates a substrate 402C having a substantially C-shaped surface. That is, the two opposing sides 410C and 412C of the substrate 402C may be curved. The extent of the C-shape may range from a half-circle, up to the point in which the two arms almost join, leaving a very small opening. As discussed above, the two ends of the C-shape may be rounded or may be straight edged. At the extreme of almost joining and having straight edges at the two ends, this provides a gap or a slit in the transducer which may allow affixing the transducer apparatus to the body while maneuvering and positioning around a chemotherapy port which is already in place on the body.

FIG. 4D illustrates a substrate 402D having a substantially annular shaped surface. That is, the at least one concave edge 406D defines an interior opening 408D bounded on all sides by the substrate 402D. The substrate 402D may have a substantially circular, oval, ovaloid, ovoid, or elliptical shaped surface with the interior opening 408D formed therein. In some embodiments, the interior opening 408D (shown circular in FIG. 4D) may have a substantially circular, oval, ovaloid, ovoid, or elliptical shape to match that of the shape of the substrate. FIG. 4D illustrates an embodiment having a circular shaped surface with the interior opening 408D formed therein. This donut shaped array (circular) may also be fitted over a chemotherapy port which is already in place on the body. Allowing a narrow slit (from an exterior boundary of the substrate to the interior opening 408) may allow at least temporary access to the interior opening and may facilitate affixing the transducer apparatus to the body while maneuvering and positioning around the chemotherapy port. Oval, ovaloid, ovoid, or elliptical shaped transducers may function similarly. For example, an oval, ovaloid, ovoid, or elliptical shape transducer may provide a more appropriate opening to avoid covering a surgical scar or skin lesion.

FIG. 4E illustrates another substrate 402E having an irregular shaped surface. As illustrated in FIG. 4E, the irregular shaped surface of the substrate 402D is substantially U-shaped, and the substrate 402E has a scalloped edge. A scalloped edge may be used with any other transducer shapes (e.g., C-shaped, rounded V-shaped, or annular shaped) disclosed herein. An irregular shaped surface may also be used with any electrode shape present on the substrate. For example, the irregular shaped surface of the substrate 402D may match (or more nearly match) the perimeter (or expanded version of the perimeter) resulting from tracing the outline of one or more individual electrode shapes.

FIGS. 4F and 4G illustrate further embodiments of substrates 402F and 402G having a substantially U-shaped surface. As illustrated in both FIG. 4F and FIG. 4G, the ends of the two opposing sides (arms) of the U-shaped substrate that define the opening may be straight (flattened curves). The arms of the U-shape may be of any width and/or length, and, indeed, the two arms of a given substrate need not be identical. Further, as illustrated, for any embodiment, the electrodes may be of any size, shape and number, and may be the same or different on the substrate. For simplicity, the electrodes in FIGS. 4A-4E were shown as circular electrodes. In particular, the shape of the electrodes for a given shape of substrate may be selected based on availability of electrodes or may be based on a desire to optimize the filling of the surface shape of the substrate. For example, the rounded triangular electrodes shown in the U-shaped substrate of FIG. 4G could be used as the only electrode shape utilized in the circular shaped substrate illustrated in FIG. 4D to provide a higher density of electrode surface area on the surface of the circular substrate.

The structure of the transducers may take many forms. In FIG. 5A, the transducer 500A has a plurality of electrode elements 502A positioned on a substrate 504A. The substrate 504A is configured for attaching the transducer 500A to a subject's body. Suitable materials for the substrate 504A include, for example, cloth, foam, flexible plastic, and/or a conductive medical gel. The transducer 500A may be affixed to the subject's body via the substrate 504A (e.g., via an adhesive backing and/or the conductive medical gel). The transducer may be conductive or non-conductive. FIG. 5B depicts another example of the structure of the transducer 500B. In this example, the transducer 500B includes a plurality of electrode elements 502B that are electrically and mechanically connected to one another without a substrate. In one example, electrode elements 502B are connected to each other through conductive wires 506B.

The transducers 500A and 500B may comprise arrays of substantially flat electrode elements 502A and 502B, respectively. The array of electrode elements may be capacitively coupled. In one example, the electrode elements 502A and 502B are non-ceramic dielectric materials positioned over a plurality of flat conductors. Examples of non-ceramic dielectric materials positioned over flat conductors include polymer films disposed over pads on a printed circuit board or over flat pieces of metal. In another example, the electrode elements 502A and 502B are ceramic elements.

FIG. 6 depicts an example configuration of one pair of transducers 602 and 604. In this example, the apparatus 600 comprises transducers 602 and 604. The first transducer 602 includes 13 electrode elements 606 which are positioned on a substrate 608 and electrically and mechanically connected to one another through a conductive wiring 610. Similarly, the second transducer 604 includes 14 electrode elements 612 which are positioned on a substrate 614 and electrically and mechanically connected to one another through a conductive wiring 616. The substrate 614 may be substantially U-shaped (as shown), C-shaped, rounded V-shaped, or annular shaped. Although the outline of the substrate 614 is depicted with straight lines and sharp vertices in FIG. 6, the outline may be curved as, for example, in FIG. 4A or 4B. The two substrates 608 and 614 may have the same, mirror image, or different shapes from each other. The transducers 602 and 604 may be connected to an AC voltage generator 618 and a controller 620. The AC voltage generator 618 is adapted to be coupled to the transducers 602 and 604 and is capable of providing a first voltage to the transducer 602 and a second voltage to the transducer 604. The controller 620 may include one or more processors and memory accessible by the one or more processors. The memory may store instructions that, when executed by the one or more processors, control the AC voltage generator 618 to induce an electric field between the two transducers 602 and 604.

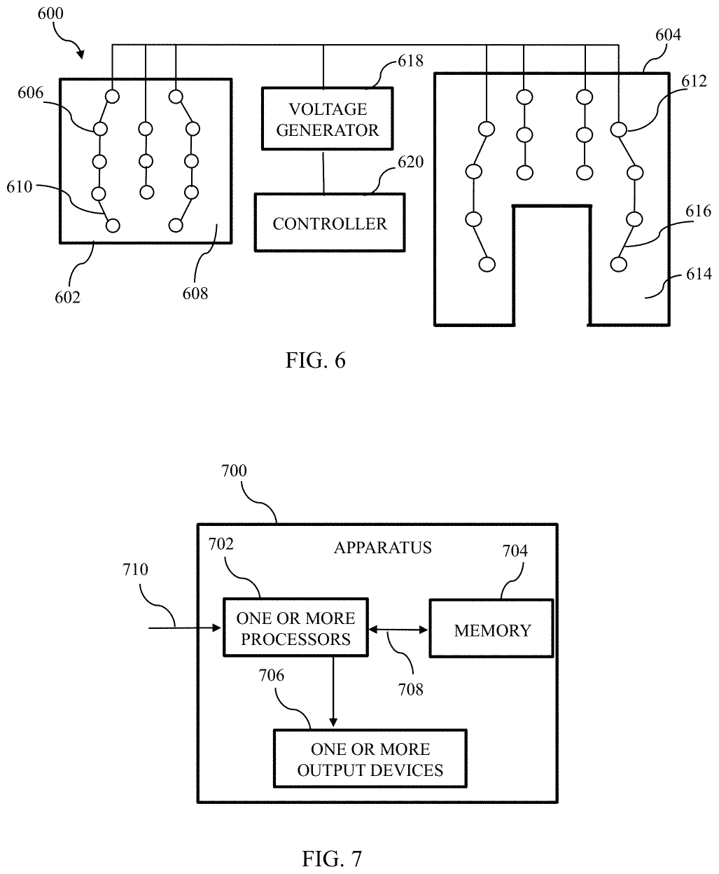

FIG. 7 depicts an exemplary apparatus to determine locations and shapes of transducers for applying TTFields. In this example, the apparatus 700 includes one or more processors 702, a memory 704, and one or more output devices 706. The memory 704 is accessible by the one or more processors 702 via a link 708 so the one or more processors 702 can read information from and write information to the memory 704. The memory 704 stores instructions that, when executed by the one or more processors 702, cause the apparatus 700 to perform one or more methods disclosed herein. The one or more processors 702 may receive inputs 710 (e.g., user inputs of image data) and, based on the inputs 710, make one or more recommendations of transducer shapes and/or locations to a user, which are output by the output devices 706.

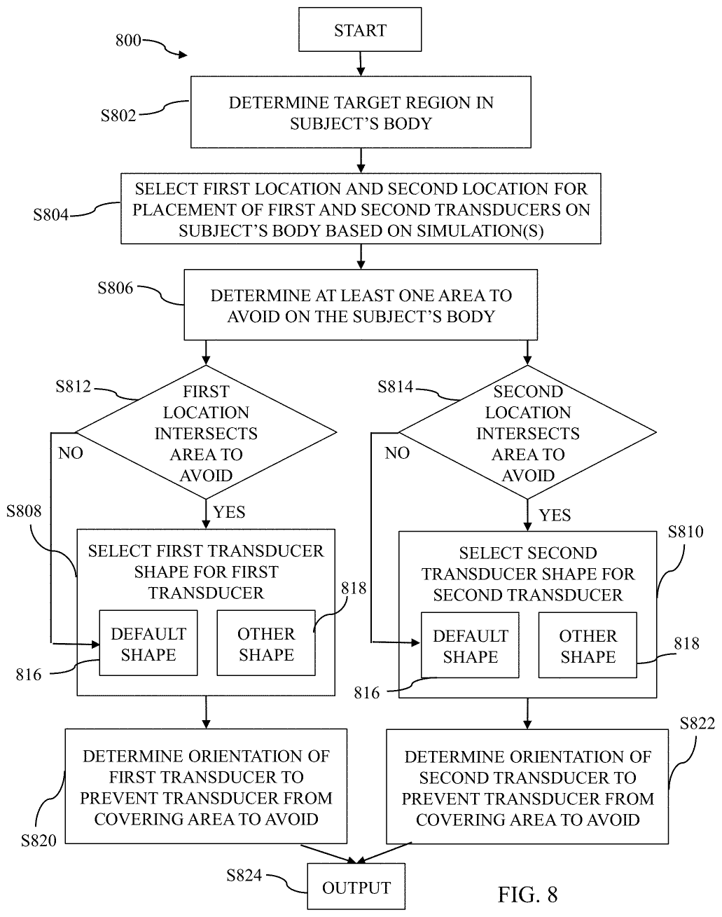

FIG. 8 is a flowchart depicting an example method 800 of determining the shape and placement of transducers on a subject's body for applying TTFields. As illustrated, the method 800 may begin at step S802 with determining a target region in the subject's body. This may be accomplished by, for example, analyzing one or more sets of image data (e.g., magnetic resonance imaging (MRI) data, computer tomographic (CT) data, etc.) to determine an approximate location and/or 3D volume of the target region in the subject's body.

At step S804, the method 800 includes selecting a first location on the subject's body for placement of a first transducer and a second location on the subject's body for placement of a second transducer, based on one or more simulations of an electric field distribution through the target region in the subject's body. This may involve, for example, performing one or more simulations (using a simulation algorithm) of the expected electric field distribution through the target region of the subject's body based on image data associated with the subject's body. More particularly, the determination may be made by comparing simulations for different possible transducer location pairs, and ranking/recommending a pair of transducer locations based on expected electric field distributions through the target region.

At step S806, the method 800 includes determining at least one area to avoid for placement of a transducer array on the subject's body. The at least one area to avoid may correspond to an anatomical feature of the subject's body or a device located in the subject's body, as discussed above. The at least one area to avoid may be determined, for example, via an image processing module identifying landmarks (e.g., anatomical features and/or devices) depicted in one or more images included with image data of the subject's body. The image processing module may use one or more object identification and/or tracking algorithms to determine/detect the locations of one or more landmarks. In another example, the at least one area to avoid may be identified based on user inputs including, for example, an indication of the presence and/or approximate location of a chemotherapy port in the subject's body, body measurements (e.g., breast size measurements), and others.

At step 5808, the method 800 includes selecting a first transducer shape for the first transducer based on the first location and the at least one area to avoid on the subject's body. Similarly, at step 5810, the method 800 includes selecting a second transducer shape for the second transducer based on the second location and the at least one area to avoid on the subject's body. The second transducer shape may be the same shape as, a mirror image shape of, or a different shape from, the first transducer shape.

Selecting the first transducer shape may involve, for example, determining at step S812 whether the first location overlaps at least a portion of the at least one area to avoid. Similarly, selecting the second transducer shape may involve determining at step 5814 whether the second location overlaps at least a portion of the at least one area to avoid. Upon determining that either of the first location or second location does not overlap at least a portion of the at least one area to avoid (“NO”), then a default transducer shape 816 may be selected for the corresponding first transducer (S808) or second transducer (S810). The default transducer shape 816 may be a rectangular, substantially rectangular with rounded corners, circular, oval, ovaloid, ovoid, or elliptical shape. Upon determining that either of the first location or second location overlaps at least a portion of the at least one area to avoid (“YES”), then a shape 818 other than the default shape 816 may be selected for the corresponding first transducer (S808) or second transducer (S810). The other shape 818 may include a substantially C-shape, U-shape, rounded V-shape, or annular shape. In some embodiments, the method 800 may include selecting the particular other shape 818 based on factors such as, for example, the relative size and position of the overlapping portion of the at least one area to avoid compared to a default shaped transducer located at the same position on the subject's body.

In an example, at step S820 the method 800 may further include determining an orientation of the first transducer having the first transducer shape at the first location of the subject's body to prevent the first transducer from covering at least a portion of the at least one area to avoid located at the first location. Similarly, at step S822, the method 800 may further include determining an orientation of the second transducer having the second transducer shape at the second location of the subject's body to prevent the second transducer from covering at least a portion of the at least one area to avoid located at the second location.

At step S824, the method 800 includes outputting the recommended first transducer shape, first location, second transducer shape, and second location to a user (e.g., via an output on a user interface). Step S824 may also include outputting a recommended orientation of one or both of the first transducer and the second transducer to the user. The outputs may be in the form of visual notifications for transducer array placement. That is, the one or more recommended placement positions for the one or more transducer arrays and the one or more areas to avoid may be displayed to the user. The notification may visually instruct the user where to place a transducer array to 1) avoid the one or more areas to avoid on the subject's body, and 2) receive an optimized electric field applied to the target region.

The invention includes other illustrative embodiments, such as the following, and any combination of these illustrative embodiments (or portions thereof) may be made to define an embodiment.

Illustrative Embodiment 1: A transducer apparatus for delivering tumor treating fields to a subject's body, the transducer apparatus comprising: a substrate; and an array of electrodes disposed on the substrate, the array configured to be positioned over the subject's body with a face of the array facing the subject's body; wherein, when viewed from a direction perpendicular to the face of the array, the substrate has at least one concave edge forming at least a portion of a boundary of the array, the at least one concave edge defines an opening between two opposing sides of the substrate, no electrodes are present in the opening, and the substrate has a substantially C-shaped, U-shaped, rounded V-shaped, or annular shaped surface.

Illustrative Embodiment 2: The transducer apparatus of Illustrative Embodiment 1, wherein when viewed from the direction perpendicular to the face of the array: the substrate has a substantially C-shaped surface, and the two opposing sides of the substrate are curved.

Illustrative Embodiment 3. The transducer apparatus of Illustrative Embodiment 1, wherein when viewed from the direction perpendicular to the face of the array: the substrate has a substantially U-shaped or rounded V-shaped surface, and the two opposing sides of the substrate are substantially straight and extend in substantially parallel directions, or deviate from being parallel by less than 45°.

Illustrative Embodiment 4: The transducer apparatus of Illustrative Embodiment 1, wherein when viewed from the direction perpendicular to the face of the array: the substrate has a substantially annular shaped surface, the at least one concave edge defines an interior opening bounded on all sides by the substrate, and the substrate has a substantially circular, oval, ovaloid, ovoid, or elliptical shaped surface with the interior opening formed therein.

Illustrative Embodiment 5: The transducer apparatus of Illustrative Embodiment 4, wherein the substantially circular, oval, ovaloid, ovoid, or elliptical shaped surface has a slit running from an exterior boundary of the substrate to the interior opening allowing at least a temporary opening in the annular shape.

Illustrative Embodiment 6: The transducer apparatus of Illustrative Embodiment 1, wherein when viewed from the direction perpendicular to the face of the array: when a straight line is drawn through the two opposing sides of the substrate, from outer substrate boundary to opposing outer substrate boundary, and across the opening, a distance along the line across the opening is at least 25% of a distance along the line across one of the opposing sides of the substrate.

Illustrative Embodiment 7: The transducer apparatus of Illustrative Embodiment 1, wherein when viewed from the direction perpendicular to the face of the array: the substrate has a scalloped edge.

Illustrative Embodiment 8: An assembly for applying tumor treating fields to a subject's body while avoiding at least one area with an anatomic feature or device, the assembly comprising: a first transducer array comprising a plurality of electrodes, the first transducer array having a first surface that is flexible for contouring to the subject's body, wherein: when viewed in a direction perpendicular to the first surface: the first surface is substantially C-shaped, U-shaped, rounded V-shaped, or annular shaped, wherein, when C-shaped, U-shaped, or rounded V-shaped, two opposing portions of the first surface are spaced apart from each other and connected to each other by a concave edge of the first surface; and, when annular shaped, a concave edge defines an interior opening bounded on all sides by the first surface; and a second transducer array comprising a plurality of electrodes, the second transducer array having a second surface that is flexible for contouring to the subject's body, wherein the second surface has the same or mirror image shape, or a different shape, than the first surface.

Illustrative Embodiment 9: The assembly of Illustrative Embodiment 8, wherein when viewed in a direction perpendicular to the second surface, the second surface has a substantially convex shape.

Illustrative Embodiment 10: The assembly of Illustrative Embodiment 9, wherein when viewed in the direction perpendicular to the second surface, the second surface has a rectangular, substantially rectangular with rounded corners, circular, oval, ovaloid, ovoid, or elliptical shape.

Illustrative Embodiment 11: The assembly of Illustrative Embodiment 8, wherein when viewed in the direction perpendicular to the first surface, the first surface has an irregular shape.

Illustrative Embodiment 12: The assembly of Illustrative Embodiment 8, wherein the first surface of the first transducer array is substantially C-shaped, U-shaped, or rounded V-shaped and is adapted to be positioned on the subject's body such that the two opposing portions of the first surface are spaced apart to straddle a first location on the subject's body having a chemotherapy port, and no electrodes of the first transducer array are located over the chemotherapy port.

Illustrative Embodiment 13: The assembly of Illustrative Embodiment 8, wherein the first surface of the first transducer array is substantially annular shaped and is adapted to be positioned on the subject's body such that the interior opening coincides with a first location on the subject's body having a chemotherapy port, and no electrodes of the first transducer array are located over the chemotherapy port.

Illustrative Embodiment 14: The assembly of Illustrative Embodiment 8, further comprising: a third transducer array comprising a plurality of electrodes, the third transducer array having a third surface that is flexible for contouring to the subject's body, the third surface having a different shape than the first surface; and a fourth transducer array comprising a plurality of electrodes, the fourth transducer array having a fourth surface that is flexible for contouring to the subject's body, the fourth surface having a different shape than the first surface.

Illustrative Embodiment 15: A computer-implemented method to determine a shape and placement of transducers on a subject's body for applying tumor treating fields, the computer comprising one or more processors and memory accessible by the one or more processors, the memory storing instructions that when executed by the one or more processors cause the computer to perform the method, the method comprising: determining a target region in the subject's body to apply tumor treating fields; selecting a first location on the subject's body for placement of a first transducer and a second location on the subject's body for placement of a second transducer, based on one or more simulations of an electric field distribution through the target region in the subject's body; determining at least one area to avoid placement of at least a portion of a transducer array on the subject's body, the at least one area to avoid corresponding to an anatomical feature of the subject's body or a device located in the subject's body; and selecting a first transducer shape for the first transducer based on the first location and the at least one area to avoid on the subject's body; selecting a second transducer shape for the second transducer based on the second location and the at least one area to avoid on the subject's body, the second transducer shape being the same or mirror image shape, or different shape from the first transducer shape; and outputting the first transducer shape, the first location, the second transducer shape, and the second location to a user.

Illustrative Embodiment 16: The computer-implemented method of Illustrative Embodiment 15, wherein the first transducer shape comprises a substantially C-shape, U-shape, rounded V-shape, or annular shape.

Illustrative Embodiment 17: The computer-implemented method of Illustrative Embodiment 16, wherein the second transducer shape is a rectangular, substantially rectangular with rounded corners, circular, oval, ovaloid, ovoid, or elliptical shape.

Illustrative Embodiment 18: The computer-implemented method of Illustrative Embodiment 15, wherein the at least one area to avoid comprises at least one of: a breast of the subject's body, a nipple of the subject's body, an ear of the subject's body, an eye of the subject's body, a surgical scar of the subject's body, a lesion on the subject's body, an armpit of the subject's body, or a chemotherapy port disposed in the subject's body.

Illustrative Embodiment 19: The computer-implemented method of Illustrative Embodiment 15, further comprising: determining an orientation of the first transducer having the first transducer shape at the first location of the subject's body to prevent the first transducer from covering at least a portion of the at least one area to avoid located at the first location.

Illustrative Embodiment 20: The computer-implemented method of Illustrative Embodiment 15, further comprising: determining whether either of the first location or the second location overlaps at least a portion of the at least one area to avoid; selecting a default transducer shape for either of the first transducer or the second transducer upon determining that the corresponding first location or second location does not overlap at least a portion of the at least one area to avoid; and selecting a shape other than the default transducer shape for either of the first transducer or the second transducer upon determining that the corresponding first location or second location overlaps at least a portion of the at least one area to avoid.

Illustrative Embodiment 21: The transducer apparatus of Illustrative Embodiment 1, wherein the substrate has a surface area between approximately 20 and 600 cm². A transducer apparatus of this size may be placed on the torso of a subject's body.

Illustrative Embodiment 22: The transducer apparatus of Illustrative Embodiment 1, wherein the substrate has a surface area between approximately 20 and 300 cm². A transducer apparatus of this size may be placed on the head of a subject's body.

Illustrative Embodiment 23: The assembly of Illustrative Embodiment 8, wherein one or both of the first surface and the second surface has a scalloped edge.

Illustrative Embodiment 24: The assembly of Illustrative Embodiment 8, further comprising a voltage generator adapted to be coupled to the first transducer array and the second transducer array and capable of providing a first voltage to the first transducer array and a second voltage to the second transducer array.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof

What is claimed is:

1. A transducer apparatus for delivering tumor treating fields to a subject's body, the transducer apparatus comprising:
a substrate; and
an array of electrodes disposed on the substrate, the array configured to be positioned over the subject's body with a face of the array facing the subject's body;
wherein, when viewed from a direction perpendicular to the face of the array,
the substrate has at least one concave edge forming at least a portion of a boundary of the array,
the at least one concave edge defines an opening between two opposing sides of the substrate,
no electrodes are present in the opening, and
the substrate has a substantially C-shaped, U-shaped, rounded V-shaped, or annular shaped surface.

2. The transducer apparatus of claim 1, wherein when viewed from the direction perpendicular to the face of the array:
the substrate has a substantially C-shaped surface, and
the two opposing sides of the substrate are curved.

3. The transducer apparatus of claim 1, wherein when viewed from the direction perpendicular to the face of the array:
the substrate has a substantially U-shaped or rounded V-shaped surface, and
the two opposing sides of the substrate are substantially straight and extend in substantially parallel directions, or deviate from being parallel by less than 45°.

4. The transducer apparatus of claim 1, wherein when viewed from the direction perpendicular to the face of the array:
the substrate has a substantially annular shaped surface,
the at least one concave edge defines an interior opening bounded on all sides by the substrate, and
the substrate has a substantially circular, oval, ovaloid, ovoid, or elliptical shaped surface with the interior opening formed therein.

5. The transducer apparatus of claim 4, wherein the substantially circular, oval, ovaloid, ovoid, or elliptical shaped surface has a slit running from an exterior boundary of the substrate to the interior opening allowing at least a temporary opening in the annular shape.

6. The transducer apparatus of claim 1, wherein when viewed from the direction perpendicular to the face of the array:
when a straight line is drawn through the two opposing sides of the substrate, from outer substrate boundary to opposing outer substrate boundary, and across the opening, a distance along the line across the opening is at least 25% of a distance along the line across one of the opposing sides of the substrate.

7. The transducer apparatus of claim 1, wherein when viewed from the direction perpendicular to the face of the array:
the substrate has a scalloped edge.

8. An assembly for applying tumor treating fields to a subject's body while avoiding at least one area with an anatomic feature or device, the assembly comprising:
a first transducer array comprising a plurality of electrodes, the first transducer array having a first surface that is flexible for contouring to the subject's body, wherein:
when viewed in a direction perpendicular to the first surface:
the first surface is substantially C-shaped, U-shaped, rounded V-shaped, or annular shaped, wherein, when C-shaped, U-shaped, or rounded V-shaped, two opposing portions of the first surface are spaced apart from each other and connected to each other by a concave edge of the first surface; and, when annular shaped, a concave edge defines an interior opening bounded on all sides by the first surface; and
a second transducer array comprising a plurality of electrodes, the second transducer array having a second surface that is flexible for contouring to the subject's body, wherein the second surface has the same or mirror image shape, or a different shape, than the first surface.

9. The assembly of claim 8, wherein when viewed in a direction perpendicular to the second surface, the second surface has a substantially convex shape.

10. The assembly of claim 9, wherein when viewed in the direction perpendicular to the second surface, the second surface has a rectangular, substantially rectangular with rounded corners, circular, oval, ovaloid, ovoid, or elliptical shape.

11. The assembly of claim 8, wherein when viewed in the direction perpendicular to the first surface, the first surface has an irregular shape.

12. The assembly of claim 8, wherein the first surface of the first transducer array is substantially C-shaped, U-shaped, or rounded V-shaped and is adapted to be positioned on the subject's body such that the two opposing portions of the first surface are spaced apart to straddle a first location on the subject's body having a chemotherapy port, and no electrodes of the first transducer array are located over the chemotherapy port.

13. The assembly of claim 8, wherein the first surface of the first transducer array is substantially annular shaped and is adapted to be positioned on the subject's body such that the interior opening coincides with a first location on the subject's body having a chemotherapy port, and no electrodes of the first transducer array are located over the chemotherapy port.

14. The assembly of claim 8, further comprising:
a third transducer array comprising a plurality of electrodes, the third transducer array having a third surface that is flexible for contouring to the subject's body, the third surface having a different shape than the first surface; and
a fourth transducer array comprising a plurality of electrodes, the fourth transducer array having a fourth surface that is flexible for contouring to the subject's body, the fourth surface having a different shape than the first surface.

15. A computer-implemented method to determine a shape and placement of transducers on a subject's body for applying tumor treating fields, the computer comprising one or more processors and memory accessible by the one or more processors, the memory storing instructions that when executed by the one or more processors cause the computer to perform the method, the method comprising:
determining a target region in the subject's body to apply tumor treating fields;
selecting a first location on the subject's body for placement of a first transducer and a second location on the subject's body for placement of a second transducer, based on one or more simulations of an electric field distribution through the target region in the subject's body;
determining at least one area to avoid placement of at least a portion of a transducer array on the subject's body, the at least one area to avoid corresponding to an anatomical feature of the subject's body or a device located in the subject's body; and
selecting a first transducer shape for the first transducer based on the first location and the at least one area to avoid on the subject's body;
selecting a second transducer shape for the second transducer based on the second location and the at least one area to avoid on the subject's body, the second transducer shape being the same or mirror image shape, or different shape from the first transducer shape; and
outputting the first transducer shape, the first location, the second transducer shape, and the second location to a user.

16. The computer-implemented method of claim 15, wherein the first transducer shape comprises a substantially C-shape, U-shape, rounded V-shape, or annular shape.

17. The computer-implemented method of claim 16, wherein the second transducer shape is a rectangular, substantially rectangular with rounded corners, circular, oval, ovaloid, ovoid, or elliptical shape.

18. The computer-implemented method of claim 15, wherein the at least one area to avoid comprises at least one of: a breast of the subject's body, a nipple of the subject's body, an ear of the subject's body, an eye of the subject's body, a surgical scar of the subject's body, a lesion on the subject's body, an armpit of the subject's body, or a chemotherapy port disposed in the subject's body.

19. The computer-implemented method of claim 15, further comprising:
determining an orientation of the first transducer having the first transducer shape at the first location of the subject's body to prevent the first transducer from covering at least a portion of the at least one area to avoid located at the first location.

20. The computer-implemented method of claim 15, further comprising:
determining whether either of the first location or the second location overlaps at least a portion of the at least one area to avoid;
selecting a default transducer shape for either of the first transducer or the second transducer upon determining that the corresponding first location or second location does not overlap at least a portion of the at least one area to avoid; and
selecting a shape other than the default transducer shape for either of the first transducer or the second transducer upon determining that the corresponding first location or second location overlaps at least a portion of the at least one area to avoid.
